(19) United States
(12) Patent Application Publication
(10) Pub. No.: US 2005/0054906 A1
(43) Pub. Date: Mar. 10, 2005

(54) Spatial detectors for in-vivo measurement of bio chemistry

(76) Inventors: Joseph Page, La Jolla, CA (US); James Plante, Del Mar, CA (US)

Correspondence Address: Joseph Page, PO Box 757, La Jolla, CA, 92038, US

(21) Appl. No.: 10/656,373

(22) Filed: Sep. 8, 2003

(51) Int. Cl. A61B 5/00

(52) U.S. Cl. 600/310

(57) ABSTRACT

Acoustic detectors having special arrangements and shapes which favor receipt of energy from a particular direction are presented for use in bio chemical concentration measurement. These detectors may include those which aim to receive acoustic energy from a portion of tissue lying slightly below the surface where interstitial fluid containing glucose and glucose indicators may be probed with middle infrared electromagnetic radiation. These devices may be arranged to listen not only to a particular direction in the tissue, but additionally to a particular chosen point. In some versions, acoustic detectors are formed of concentric ring elements having geometries which facility constructive interference between successive wave fronts of a received acoustic wave. Alternatively, spherically shaped reflecting surfaces may be used as a lens to couple the energy emitted from interstitial fluid to a transducer which lies at or near the skin surface.

### BACKGROUND OF THESE INVENTIONS

1. Field

The following inventions disclosure is generally concerned with systems for non-invasive, in-vivo measurement of blood analytes and other bio chemistry, and more specifically concerned with systems having detection apparatus arranged to preferentially receive acoustic energy from selected locations; i.e. detectors having spatial or directional bias.

2. Prior Art

Advanced detection of bio chemical states is useful in the general practice of medicine. These inventions are intended to apply to many different circumstances where quantification of bio chemical states is useful. However, it is more illustrative to direct the discussions following to a single particular disease where one tends to more fully appreciate the nature of benefits. These benefits will similarly be found in other areas of medicine and health control.

Reportedly, 120 million people suffer from diabetes mellitus. This number is expected to double in the next 10 years. Both quality of life and lifetime are dramatically affected by the disease. Diabetes affects not only internal organs, circulation and eyesight, but additionally life and limb.

Great relief comes to those diabetics who monitor and maintain their blood glucose concentration within a prescribed manner. As such, systems for use in determination of blood glucose levels are in great demand. The availability, ease of use, and precision of these systems will require advances for many years to come. Every incremental improvement in these systems brings about monumental benefit in quality as well as extent of life. Accordingly, systems arranged to quantify biochemical states, blood glucose concentrations, for example.

In-Vivo Glucose Measurement Systems

Sophisticated equipment which can be used to precisely determine the concentration of glucose in blood is readily available in hospitals and patient treatment centers. This equipment is complex, large, difficult to operate and expensive.

Portable kits operable by patients ‘in-the-field’ are also quite readily available. However, these systems require that blood be first removed from a person. In a commonly used procedure, a person sticks his/her finger to remove a drop of blood to be placed on a chemical strip. This technique is accompanied by pain and discomfort, yet it remains the dominant method today because it is very reliable. Even where micro-fine lances have been promoted, the technique remains painful. A typical patient's response is to test less frequently. Less frequent testing results in less glucose concentration control and damaging health effects.

Alternative non-invasive or minimally invasive techniques have also been proposed. A device sold under the mark ‘Glucowatch’ uses electrophoresis to extract fluid from tissue through the skin and into a chemical sensor. These devices remain bulky, unreliable, and inaccurate for some users.

An army of researchers continue to develop other non-invasive systems which might replace the finger-stick based systems. One leading technique relates to an optical stimulation of glucose molecules directly producing a measurable acoustic response. Methods sometimes called ‘photoacoustic effect spectroscopy’ are included in these types of non-invasive technique.

Photoacoustics Effect Spectroscopy

‘Photoacoustic effect spectroscopy’, ‘PA’, includes techniques of optically exciting thermal activity in matter and examining resulting pressure waves generated in response thereto. In essence, light of a well selected wavelength is injected to a sample being tested which may be comprised of atomic or molecular matter of interest. The wavelength of light is tuned to natural resonances of molecules for which presence is being tested. Interaction between light and matter causes optical energy to be converted to vibrational energy, and very quickly thereafter, heat energy. By applying a plurality of quick pulses, a pressure wave is formed which is readily detected with acoustic transducers.

PA in Blood Glucose Measurements

Photoacoustic effect spectroscopy has been applied to test for the concentration of glucose in blood. Generally, this involves use of a so-called ‘IR window’ in tissue. Light having wavelengths in the near infrared NIR region of the spectrum, that is from about 0.8 microns to about 2.5 microns, easily propagates in tissue and may interact with glucose molecules to stimulate heating thereof. Great difficulties are found in these systems. The range of wavelengths which cause glucose to respond is quite broad and there are many spectral bands which will activate vibration in the glucose molecules. Further, these same wavelengths similarly interact with other tissue constituents. Thus, the noise in these systems is very nearly as large in magnitude as the signal produced leaving the accuracy of such techniques in question. Many factors necessarily present in a living human have a tendency to upset the results of such glucose testing. These include sweat, temperature, vibration, inhomogeneous tissue, among many others.

Longwave PA for Glucose Detection

Light of the nature described as belonging to the middle infrared spectra MIR may be used to more accurately measure glucose concentrations. Middle infrared light may be far more suitable for stimulating particular vibrations in the glucose molecules. In other words, natural glucose resonances are sharper and greater in magnitude in the energy bands which correspond to these wavelengths. Thus, one is able to generate an acoustic signal many times greater in magnitude when using MIR than NIR. Indeed, this has been well demonstrated by Quan et al, Phys. Med. Biol. 38 (1993) 1922-1922, Glucose determination by a pulsed photoacoustic technique. A tissue phantom was used to prove the effectiveness of the technique. While great results were produced, using large lasers in a laboratory environment is not convenient for use by diabetics not having expertise in the operation of heavy laboratory equipment.

A very special alternative form of MIR-PA is taught first and presented by v. Lilienfeld-Toal of Elté Sensorics in Germany in U.S. Pat. No. 6,484,044. V. Lilienfeld-Toal teaches of a new, high power semiconductor laser which does not require any special skill to be used. It is very small and lightly consumes energy as its efficiency is comparatively high. Consequently, these systems may be made as compact and miniaturized devices and be used easily by unskilled patients. Production units might be as small as a few hundred cubic centimeters and operate as a desktop model in a common household environment without special supporting apparatus.

The advantages associated with MIR-PA do not come without difficulty. MIR light is quickly absorbed and does not propagate well in tissue. It is not a trivial task to get a light beam into the portion of tissue where glucose molecules may be found. Since glucose is not found in the outer layer of the skin at appreciable levels, or in levels which indicate concentration of glucose in the blood, light must be of sufficient strength to penetrate the top surface of the skin. However, most light is absorbed in the epidermis producing a very large acoustic signal which interferes with signals produced by glucose. It would be quite useful to realize a detector which discriminates against acoustic signals produced in the epidermis.

Intracellular and interstitial fluids which are found quite near the skin surface do contain glucose in levels which reflect blood glucose concentration. Glucose molecules in the interstitial fluids are good targets for PA systems based on MIR light. MIR light of sufficient strength can penetrate the outer layers of skin and interact with interstitial fluids.

High Performance Detectors

The MIR-PA systems of above described arts suffer for want of precision in the detection step. A great portion of the detected acoustic wave is produced by heating of matter which does not include glucose molecules. This is a direct result of the fact that v. Lielienfeld's detector is equally coupled to all regions of the tissue. Acoustic waves produced at the skin arrive at the detector and cause strong electrical response. The transducers of von Lielenfeld tend to ‘see’ all tissue regions equally; that is, those transducers are strongly coupled to activity which is not useful in the glucose concentration measurement.

Accordingly, it would provide significant advantage to have systems where the detection transducer is focused upon and directed towards primarily the regions of tissue known to yield PA activity in response to glucose being stimulated by MIR light energy. Detectors which are highly coupled to tissue containing interstitial fluids and glucose molecules, but discriminative of tissue not containing substantial amounts of glucose presents measurement advantage and accuracy which cannot be found in the art.

Notwithstanding, techniques have been discovered which provide very novel uses of non-invasive, in-vivo glucose detection systems, particularly with respect to those based upon photoacoustic spectroscopy with MIR illumination sources further having sophisticated directional detectors. In contrast to the good and useful inventions of the art mentioned, each having certain features that are no less than remarkable, inventions taught here are concerned with tuned detectors which can be strongly coupled to selected matter.

### SUMMARY OF THESE INVENTIONS

Comes now, Joseph Page and James Plante with inventions of non-invasive bio chemical measurement systems including devices and methods employing detectors and detection schemes having a directional or spatial preference and bias. It is a primary function of these measurement systems to provide improved accuracy and efficiency in non-invasive chemical concentration measurements. It is a contrast to prior art methods and devices that those systems do not include directional detectors which are preferentially coupled to specific tissue regions.

Detectors including acoustic or pressure transducers are configured to yield a spatial bias with respect to sensitivity. These detectors are coupled to a particular tissue region more strongly than other nearby regions. Thus the detectors may be more strongly coupled to those tissue portions which best produce activity which indicates presence of the particular chemistry being targeted; for example a glucose molecule.

These systems include either of two classes of directional detector. In a first class, a Fresnel type lens results in constructive interference between two or more successive wavefront for selected points and destructive interference for others. In this way, the device preferentially receives energy from the design ‘target point’ while being detuned or decoupled from others. In a second class, an ‘acoustic lens’ is formed in a spherically shaped surface which reflects incoming waves in a manner causing them to become concentrated and arrive simultaneously in a small space. These detectors couple energy emanating from one point to another point remote from the first. The focus point may include the active part of a transducer. Energy from points other than the target point, tends to be dispersed away from the detector. Accordingly these detectors, so arranged, results in a system having a spatial bias; one which favors detection of acoustic energy from certain points while discriminating against detection from others.

Objectives of These Inventions

It is a primary object of these inventions to provide non-invasive chemical testing systems.

It is an object of these inventions to provide non-invasive bio chemistry testing systems based upon directional detectors.

It is a further object to provide non-invasive testing systems having detectors coupled to a specific point(s) in tissue being tested.

A better understanding can be had with reference to detailed description of preferred embodiments and with reference to appended drawings. Embodiments presented are particular ways to realize these inventions and are not inclusive of all ways possible. Therefore, there may exist embodiments that do not deviate from the spirit and scope of this disclosure as set forth by the claims, but do not appear here as specific examples. It will be appreciated that a great plurality of alternative versions are possible.

### BRIEF DESCRIPTION OF THE DRAWING FIGURES

These and other features, aspects, and advantages of the present inventions will become better understood with regard to the following description, appended claims and drawings where:

FIG. 1 is a perspective drawing of a tissue sample of interest;

FIG. 2 is a cross section drawing of a tissue sample and acoustic detector coupled therewith;

FIG. 3 is a similar cross section showing a special multi-element transducer;

FIG. 4 is a perspective drawing showing a multi-element detector coupled to tissue;

FIG. 5 illustrates a preferred version with a special access port for optical energy;

FIG. 6 shows a similar system in cross section;

FIG. 7 is a perspective drawing of a very special multi-element device;

FIG. 8 shows cooperation of an optical beam with an acoustic transducer in communication with tissue being examined;

FIG. 9 shows another advanced multi-element detector system;

FIG. 10 illustrates a special acoustic system including an acoustic reflecting element in cooperation with a crystal transducer; and

FIG. 11 shows special cooperation in the system to preferentially ‘listen’ to a particular point in the tissue.

### GLOSSARY OF SPECIAL TERMS

Throughout this disclosure, reference is made to some terms which may or may not be exactly defined in popular dictionaries as they are defined here. To provide a more precise disclosure, the following terms are presented with a view to clarity so that the true breadth and scope may be more readily appreciated. Although every attempt is made to be precise and thorough, it is a necessary condition that not all meanings associated with each term can be completely set forth. Accordingly, each term is intended to also include its common meaning which may be derived from general usage within the pertinent arts or by dictionary meaning. Where the presented definition is in conflict with a dictionary or arts definition, one should use the context of use and liberal discretion to arrive at an intended meaning. One will be well advised to error on the side of attaching broader meanings to terms used in order to more fully appreciate the depth of the teaching and to understand all the intended variations.

‘Detector’—refers to a device including subsystems and support for use in detection of acoustic energy. For example, a ‘detector’ may include transducer or transducers, electronic support such as phase delay mechanism and amplifiers, also a detector may include reflectors, arrangements of lenses such as Fresnel lens systems or diffractive lenses.

‘Transducer’—for purposes of this invention, is a device which converts acoustic energy to electric energy. Appropriate crystals, including those known as ‘piezo-electric’ crystals for example, display an effect whereby displacements in the crystalline lattice result in a voltage appearing across the device. The voltage is an indication of the degree of displacement and consequently these crystal serve well as acoustic transducers or microphones.

‘MidIR wavelengths’—are those electromagnetic spectra lying between about 3 microns and 70 microns in wavelength.

“Acoustic/audio”—‘acoustic’ or ‘audio’ energy is the energy which is embodied as pressure gradients in an elastic medium which propagate in the medium in accordance with traveling wave principles.

‘Target Point’—is used herein to describe a point within a tissue under examination. Generally, the point lies in a region containing targeted chemistry; for example in interstitial fluid or blood. A target point is in the ideal location to form a relationship with a detector's geometry whereby acoustic energy is most efficiently coupled to the detector transducer.

‘Nearby points’—A region may be comprised of a geometric center or reference and locations near the center. Nearby points are those in the region which might be similarly situated and follow similar behavior and become subject to similar exposure as the center.

### PREFERRED EMBODIMENTS OF THESE INVENTIONS

In accordance with each of the preferred embodiments of these inventions, there is provided apparatus for and methods of in-vivo measurement of bio chemistry with directionally and spatially biased, shaped detectors. It will be appreciated that each of these embodiments described include both an apparatus and method and that the apparatus and method of one preferred embodiment may be different than the apparatus and method of another embodiment. While it has been stated that these devices and methods are quite useful in detection of many forms of bio chemical substances, the following examples are primarily directed to the specific case of blood analytes including glucose and glucose markers.

Detectors are arranged with special geometry to favor receipt of energy from a particular direction or location are presented for use in glucose concentration measurement. These detectors include those which receive acoustic energy from a portion of the tissue lying slightly below the surface. In some versions, acoustic detectors are formed of concentric ring elements having geometries which facilitate constructive interference between successive wave fronts of a received acoustic wave. Alternatively, spherically shaped reflecting surfaces may be used as a lens to couple the energy emitted from interstitial fluid to a transducer which lies at or near the skin surface.

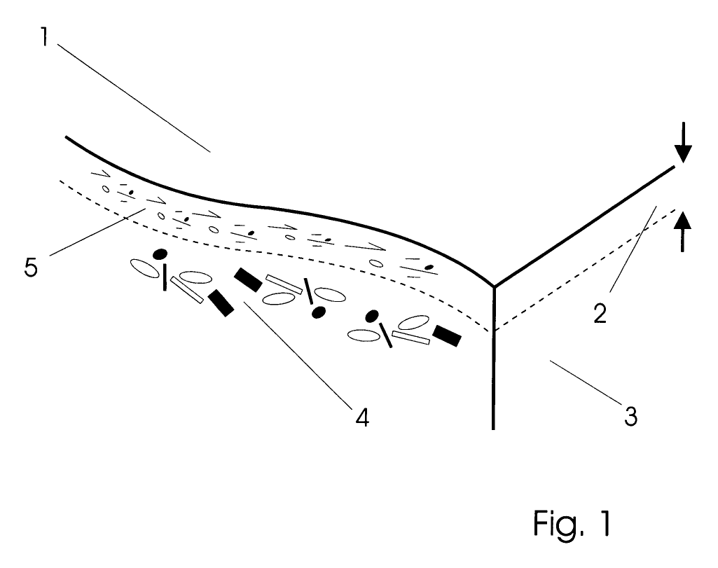

With reference to drawing FIG. 1, one can appreciate that a section of tissue has a complex composition. A top surface of the skin 1 provides an interface to which measurement systems may be readily coupled. The skin may be divided into three layers, namely, the epidermis 2, dermis 3, and subcutaneous fat, each with their own sub layers. The outermost layer of the epidermis is composed of a relatively thin, but rough, protective top layer of dead and dry skin cells, known as the ‘stratum corneum’. The remainder of the epidermis, including the stratum lucidum, stratum granulosum and stratum spinosum, is made up of cells called keratinocytes as well as melanocytes, which are pigment cells responsible for skin pigmentation. The thickness of the epidermis varies from 0.1 mm in the eyelids to nearly 1 mm on the palms and soles.

The dermis consists of a variety of cells, fibers, amorphous ground substance, nerves, oil glands, sweat glands, blood vessels and hair roots. Its upper layer is called the papillary dermis and contains the vascular network and sensory nerve endings, whereas the deeper layer, referred to as reticular dermis, consists mainly of a loose connective structure and epithelial-derived structures such as glands and follicles. The thickness of the dermis varies from 0.3 mm in the eyelids to about 3 mm in the palm and soles. Subcutaneous fat is composed of fat cells, which form a cushioning layer between the skin and the deeper muscles. It also has an abundant blood content. The dermis layer 3 has fine blood vessels, intracellular and interstitial fluids. Stippling 4 and 5 is added to respective layers to remind readers of the different composition of various tissue layers.

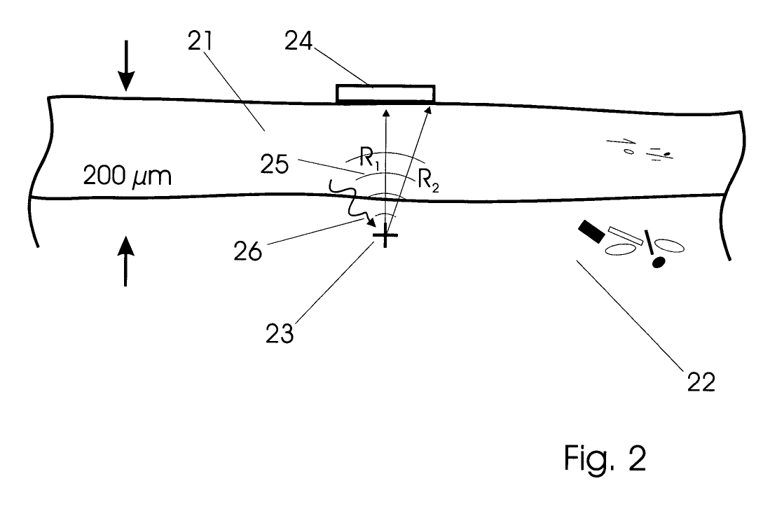

FIG. 2 illustrates a very important geometric configuration. A cross section of a tissue sample comprises a epidermal layer 21 and a dermis layer 22. A target point 23 lies deep within the skin under the surface. In some preferred versions a target point is in a region where intracellular and interstitial fluids may be found; perhaps at about 200 micrometers below the skin surface in some cases. At this location, the tissue is comprised of matter which includes glucose in concentrations which may be representative of blood glucose levels. A piezo-electric PZT crystal acoustic transducer 24 is in intimate contact with the tissue by way of it surface. It may be further coupled by way of an oil or other fluid to improve acoustic contact at the skin/PZT interface. Acoustic energy embodied as stress, or pressure waves 25, is produced as a result of long wavelength light 26 illuminating glucose molecules at the target point 23 of interest. The acoustic energy propagates toward the skin surface to fall incident upon the detector transducer. Acoustic energy released from a point of interest will form a spherical wave and propagate in all directions away from that point. While only the energy moving toward the transducer is shown, it is to be understood that energy goes in all directions.

In view of these waves having a spherical geometry, careful observation of the arrangement indicates one can expect a phase delay between energy arriving at the center of the detector with respect to energy arriving at the edge of the detector. This is due to the fact that the distance from the emission point at the detector is different for different points on the detector. It is readily understood that the distance R1 is less than the distance R2. Wavefronts will first arrive at the center of the detector and later arrive at the detector edges. It can be said that the detector doesn't match well the measurement geometry. While it is possible to form a curved detector, a detector having a shape which approximates the wavefront, such concept is accompanied by great difficulties.

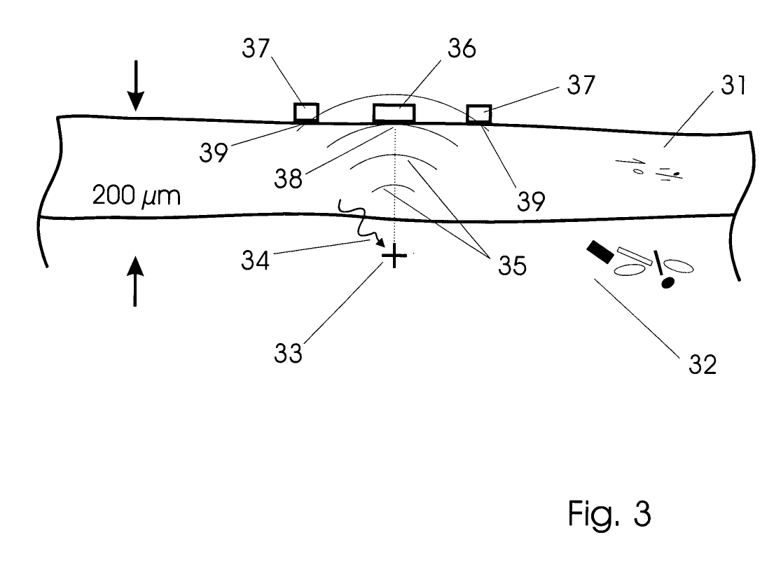

It is in agreement with these inventions that a detector be fashioned to cooperate with geometries of the measurement environment; i.e., spherical waves emitted from a point of interest deep in tissue. Accordingly, the multi-element sensor of FIG. 3 is a first preferred version having this property. Tissue layers 31 and 32 form the medium within which lies the test point of interest 33. That point is illuminated with MidIR electromagnetic energy 34. Absorption by glucose molecules causes local heating and expansion of the matter at the absorption point. Pressure waves 35 propagate away from the point and towards the surface of the skin. A multi-element detector is comprised of a center element 36, and a peripheral element 37; the reader is reminded of the axial symmetry of the system thus in cross section this element appears as two when in fact it is a single annular element. Detailed examination of the drawing suggests that for appropriately placed detector elements, successive wavefronts will fall incident simultaneously upon both elements. Wavefront 38 arrives at the center element precisely at the same time as wavefront 39 arrives at the peripheral element. Acoustic energy emitted from points nearby target point 33 will not have this property of simultaneous arrival. Thus the energy from those points will not cause the detector to respond as strongly as the detector will respond to the target point. In this way, we say the detector geometry is designed specifically to listen to one point preferentially and discriminate against all others. Experts in the field of Fresnel lens design will recognize that the concept can be extended further to include yet another element which is placed further from the symmetry axis to simultaneously receive energy from an N+2 wavefront; and so on. It is therefore fully anticipated that these multi-element detectors may be comprised of a plurality of elements which is greater than two. These drawings are directed to versions of two element devices for clarity and a better understanding.

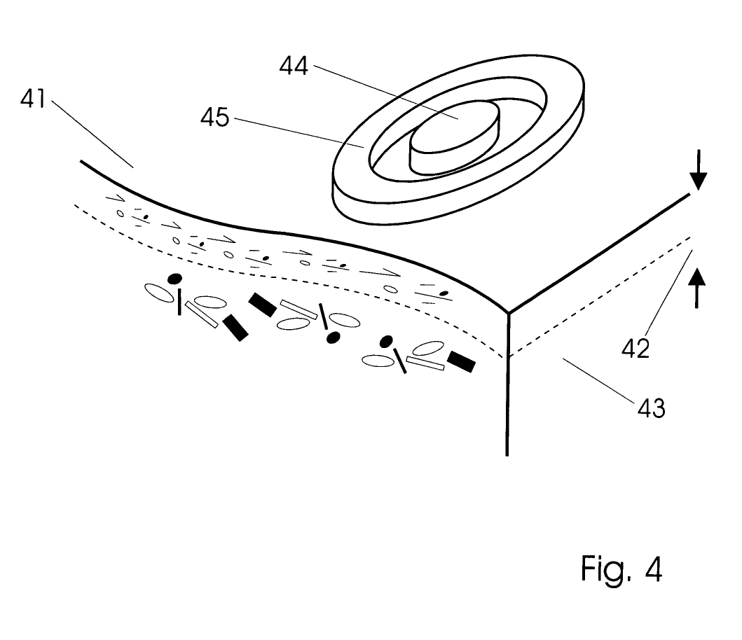

The drawing FIG. 4 shows this detector in perspective. The detector lies on the skin surface 41, the detector geometry is specifically designed with a particular depth 42 at which one may find the target point in the under layers of tissue 43. These multi-element detectors are comprised of a center element 44 and a peripheral element 45 arranged in view of acoustic wavelength such that they will address a selected point in the tissue. These elements are concentric with each other and have circular-axial symmetry; they are cylinders. The acoustic wavelength is controlled by applying multiple short optical pulses separated by a selected time period. The period of that cycle sets the acoustic wavelength and implicitly the distance between detector elements.

While FIGS. 2 and 3 seem to suggest that optical energy arrives from an oblique angle, this is not typically a good approach. Since absorption of optical energy is quite high in tissue of interest at long wavelengths, it is desirable to illuminate the test point with a beam having passed through as little tissue as possible. The best way to accomplish this is forming a more direct path for the illumination beam. Although center element 44 seems to block the direct path, a special version can accommodate an axial beam.

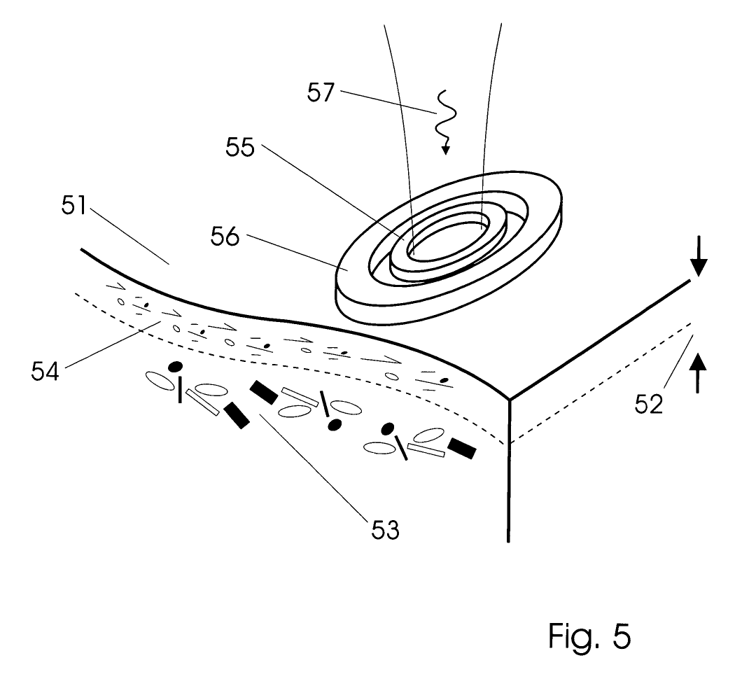

With reference to drawing FIG. 5, one can envisage a detector which accounts for an optical port at its center but maintains a multi-element axial configuration designed to be most responsive to a specific point in the tissue. (As a matter of precision, one could argue that the detector as shown actually is ‘tuned’ several points including the target point and some points lying on the symmetry axis but at N times the distance of the target point. While this is true, those points are sufficiently deep in the tissue whereby energy returned from them will have little or no impression on the primary return from the target point). Tissue sample 51 includes a target depth indicated by 52 which lies within the dermis layer 53 and below the epidermis layer 54. Multi-element transducer is comprised of a plurality of annular rings, a first ring 55 having an optical port at its center, and a second ring 56 concentric therewith the first ring and the port. In this configuration, the acoustic detector can focus on a particular point in the tissue and accommodate an input optical beam of long wavelength light 57. It is entirely possible for a multi-element transducer to include additional concentric rings; such extension is completely anticipated.

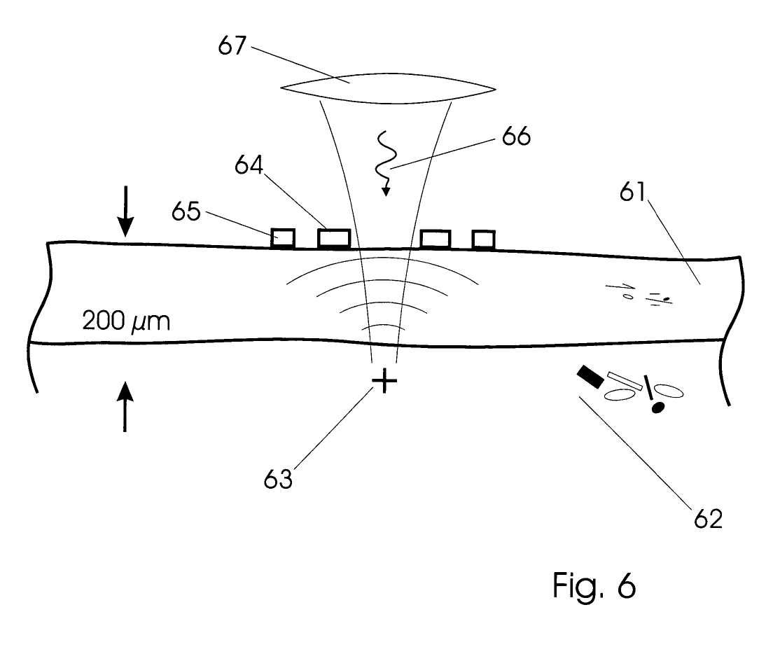

FIG. 6 illustrates a similar version in cross section. Epidermis layer 61 lies above a dermis tissue layer 62 containing interstitial fluid and further including target point 63 which may be about 200 microns beneath the surface. Acoustic transducer including inner element 64 and outer element 65 are arranged to receive acoustic energy from successive wavefronts in phase with respect to the target point. Center element has an aperture operable to permit an optical beam to pass there through. In some versions, a lens 67 or other suitable optics may be used to couple the beam to the tissue/detector arrangement. An optical beam introduced this way permits efficient transmission into the tissue and good cooperation with a detector tuned to a specific target point in the tissue.

Because tissue matter is certainly not uniform, measurements sometimes vary greatly from one tissue point to another nearby point. For this reason, it may be desirable to move the target point about a macro region of tissue to find a ‘sweet spot’; i.e. one which provides a quality return signal. It can be impractical to ask an operator to manipulate the position of a detector until a good signal is received. Feedback loops which include user actions can present considerable difficulty. It is preferable to automate the function whereby the detector ‘finds’ a measurement sweet spot. While mechanical means might be arranged to move the detector about the surface of test tissue until a sweet spot is found, special detectors of these inventions account for adjustments of the target point location via purely electronic delay means. Multi-element detectors are fashioned with radial demarcation to form a special array of acoustic elements which operate together. By applying phase controls/delays to received signals, the point within the tissue to which the detector is coupled greatest, is moved about.

A phase delay is effected by introducing delay electronically in signals produced in the transducers. PZT crystals produce a voltage across crystal surfaces in response to pressure gradients therein. Electronic circuits and amplification means may include variable delay schemes which correspond to phase delays of received acoustic waves.

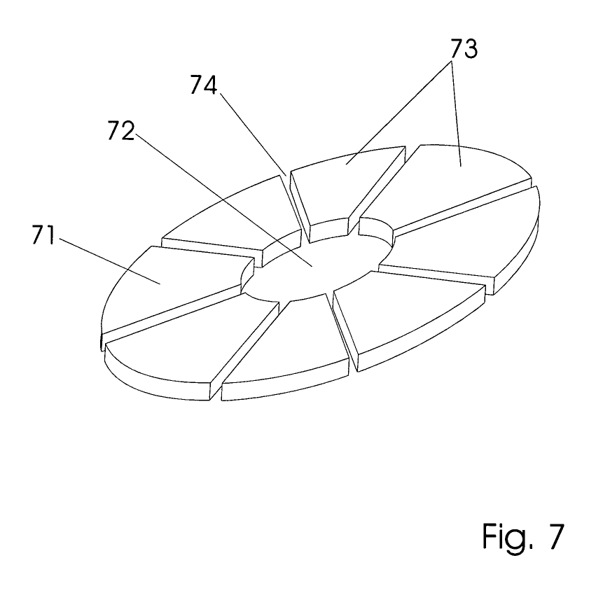

FIG. 7 illustrates a first version of these multi-element detectors having a steering function. The entire detector geometry remains circular cylindrical with an optical port at the center. A single element 71 approximates a pie wedge shape with the apex missing. The optical port 72 is fashioned with respect to the constraints on input beam size and shape. This may typically include a radius of about 100 microns. While preferred versions include those having a circular port, it is entirely anticipated that other shapes be included such as to accommodate rectangular beams. Transducers may be comprised of a plurality of identical elements 73, in this example, there are 8 symmetrically distributed identical elements which form the entire transducer array. While an eight element array is a preferred arrangement, it is not sacred by any measure. Detectors having between two and many tens or hundreds of elements may be possible. By careful manipulation of phase delay, the detector array is coupled to various points in the tissue. In other words, one can ‘steer’ the detector response to ‘listen’ to various desired points in a given tissue region. When one point is not producing acceptable return signals, the detector may automatically be switched, by phase manipulation, to look at another point where good returns may be found.

While one might argue that this steerable detector is redundant in view of the fact that no steering is provided for the illumination beam, the suggestion is not valid. Because it is anticipated that the illuminated region is quite large, partly due to significant dispersion of light in tissue, there exist many points within an illuminated field. The transducer may be directed to address any of the points within the illuminated region which is producing a strong return. Thus it is unnecessary to steer the input beam with the detector.

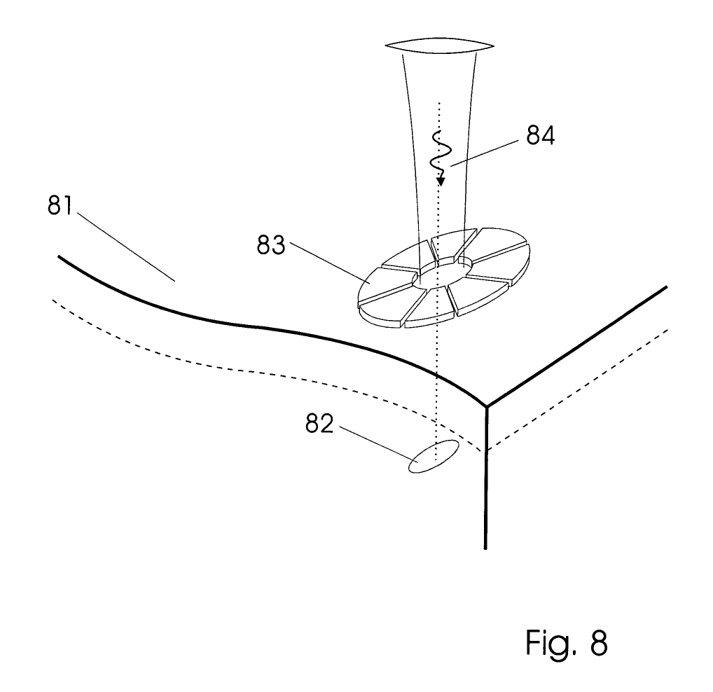

FIG. 8 shows the detector described in conjunction with a test tissue sample 81. A whole region of interest 82 deep inside the tissue may be addressed by the detector 83. Illumination beam of MidIR optical radiation 84 is directed into the tissue by way of optical port at the center of the transducer elements. Return signals from illuminated tissue propagate toward the detector elements and are received there. By applying phase delays appropriately to signals produced at various detector elements, the precise point in the addressed region may be moved from one point to another. Where the return signals are bad due to inhomogeneous tissue or other difficulty, the detector may be redirected to another point where a quality signal may be received. In some schemes, the detector may be scanned through a set of predefined points and results averaged to provide a most consistent measurement.

The detector of FIG. 8 may be insufficient for control of depth of the address plane. As in prior examples, this may be overcome by considering a detector of multiple concentric circle elements. Since the depth within the tissue at which a measurement is most reliable is more or less known, the concentric rings may be arranged to correspond accordingly.

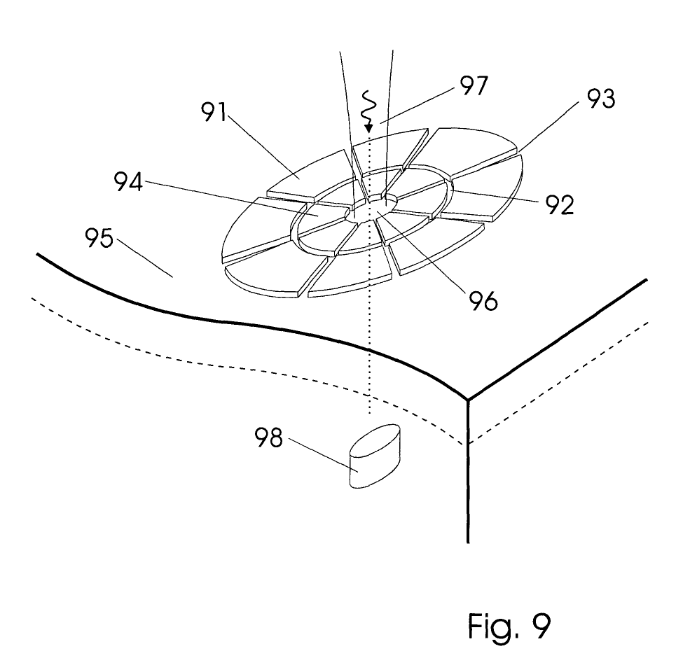

FIG. 9 illustrates a multi-element 91 detector with both axial and radial divisions, 92, 93 respectively, between elements. An inner ring of eight elements 94 is formed about an annulus concentric with an outer ring of eight similar elements. The entire detector occupies a substantially planar space and is placed in intimate contact with the surface of a test tissue, typically skin 95. The detector accounts for an optical port 96 to pass an input optical beam 97. These detectors may be used to address a large volume of target points 98.

It is not only the physical position of the concentric rings with respect to each other that contributes to define the depth of the plane being addressed, i.e. coupled to the greatest extent, but also any phase delays which may be applied between the respective concentric rings. Thus, one can not only tune the position being addressed within the plane, but also the depth of the plane within the tissue. Accordingly, this detector offers a 3-dimensional scanning technique whereby an entire volume of tissue may be examined without requiring positional adjustments of the detector on the tissue surface. The addressable volume permits averaging and tuning for highest coupling within a highly inhomogeneous tissue.

While multi-element detectors employing phase delay techniques yield excellent steerable, high-performance detectors, other systems may be employed which include detectors with directional and spatially specific coupling to improve results in a photoacoustic glucose measurement apparatus. An acoustic chamber can be constructed to have a focus point whereby energy emanating therefrom is strongly coupled to another point while energy from all other nearby points is dispersed. Thus, a shaped acoustic chamber may be used to effectively couple a specific target point with a transducer in a glucose detection system.

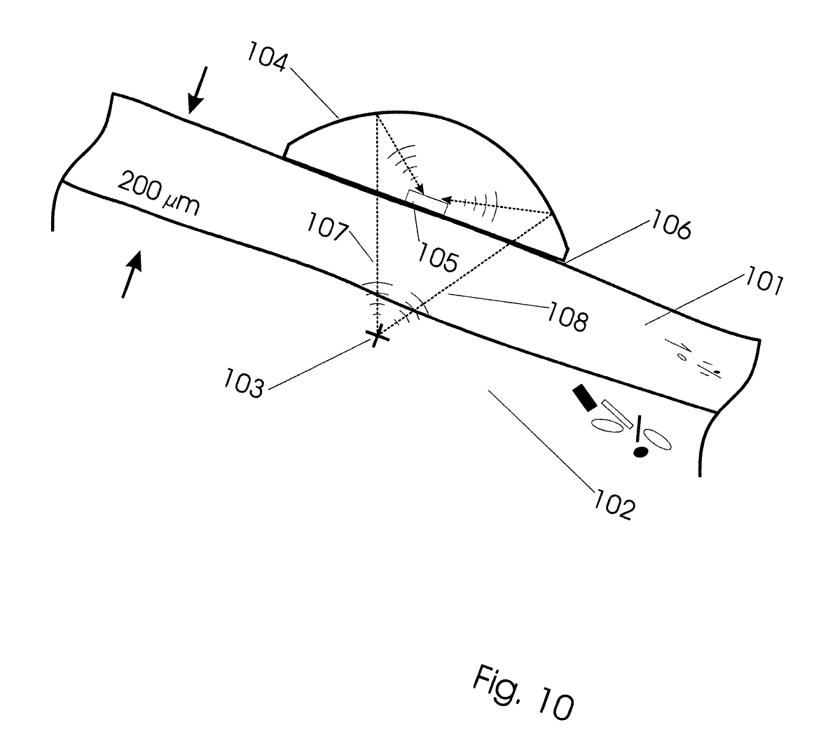

FIG. 10 shows a tissue sample of epidermis 101 and dermis 102 having therein a target point 103. Acoustic cell 104 forms a reflector from which acoustic energy is redirected and focused onto a pressure transducer 105. The curved surface of the reflector may be spherical or an aspheric such as parabolic. The acoustic cell is kept in good contact with the skin surface at a skin/cell junction 106 which may be improved with a coupling fluid to better transmit acoustic energy there through. Acoustic path 107 and path 108 are quite different in direction but both ultimately land upon the piezoelectric PZT crystal simultaneously to produce a strong displacement therein.

One might inquire as to how an optical beam might be introduced for illumination of the target point. It is possible to form acoustic cells from material, for example ZnSe, which is transparent to MIR light. Indeed, in some cases the acoustic cell forms on optical lens to further focus input light beams to the target point. An input beam incident from above is focused by the plano-convex lens/detector of FIG. 10 to arrive at the target point in high density.

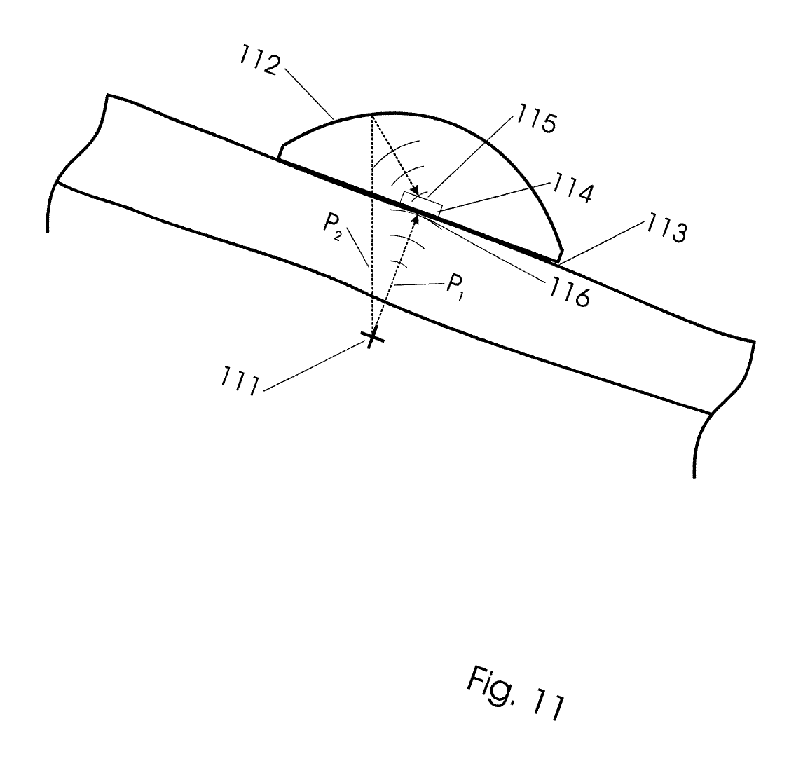

An additional improvement may include proper configuration such that the distance from the target point to the transducer is designed to cooperate with the distance from the target, to the reflector and finally to the transducer whereby the phase is adjusted to produce an additive effect with respect to the desired target point. FIG. 11 shows the paths which are important in this configuration. A direct path, P1 is a known distance from a target point 111. The design acoustic wavelength is set by the repetition rate of the optical pulses. Acoustic cell surface 112 reflects acoustic energy incident thereon towards the transducer 114. The entire arrangement lies on the skin surface to form clean junction 113 which acoustic energy easily passes. Finally, the distance of P2 is designed such that an additive effect is produced with regard to energy propagating on path P1. In the figure, the wavefronts arrive simultaneously at the transducer top 115 and the transducer bottom 116. In this way, a selected point in the tissue is maximally coupled to the detector while all other points are only weekly coupled to the sensor.

The examples above are directed to specific embodiments which illustrate preferred versions of devices and methods of these inventions. In the interests of completeness, a more general description of devices and the elements of which they are comprised as well as methods and the steps of which they are comprised is presented herefollowing.

Apparatus of These Inventions

In most general terms, apparatus of these inventions may precisely be described as devices for in-vivo determination of bio chemical concentrations having an optical illumination source and a detector. The detector includes at least one pressure transducer having a geometric shape to couple a test point more strongly than points immediately surrounding. The detector further acts as a directional ‘microphone’ whereby a predetermined space is favored over other regions with respect to transducer response and performance. These detectors are arranged to address portions of flesh containing interstitial fluids in human tissue; in some cases these portions of flesh are about 200 micrometers beneath the skin surface. In some versions, these detectors include a plurality of transducers to form a multi-element detector array. The transducers may be arranged in an axially symmetric pattern. Preferred versions include transducers which substantially form an annulus. Or, a plurality of transducers in a plurality of annuli, one concentric with another. These detectors also may include electronic means of promoting a phase delay of signals generated at either of said transducers. Detector versions include those having an optical port at the center. Further, others may include detectors of multiple rings, each ring being bifurcated into a plurality of wedge section sub-elements.

Methods of These Invention

In most general terms, methods of the inventions may precisely be described as including the steps of: illuminating a tissue sample with middle infrared light and receiving pressure waves emanating from illuminated tissue at a skin surface interface; converting those pressure waves into electronic signals at a detector shaped such that energy emitted from a particular point is coupled to the transducer with a greater efficiency than other nearby points. In preferred versions the step of ‘converting pressure waves’ is done at a plurality of spatially removed locations. In some cases, a phase delay is introduced into at least one of the electronic signals. These ‘converting pressure waves’ is done at a plurality of locations distributed about a symmetry axis. Pressure wave conversion may be done simultaneously and thereafter a phase delay is created electronically and introduced into at least one of the electronic signals.

One will now fully appreciate how accurate, in-vivo bio chemical measurements may be made with photoacoustic systems having precise detectors well coupled to specific tissue locations. Although the present inventions have been described in considerable detail with clear and concise language and with reference to certain preferred versions thereof including the best mode anticipated by the inventors, other versions are possible. Therefore, the spirit and scope of these inventions should not be limited by the description of the preferred versions contained therein, but rather by the claims appended hereto.

1) Apparatus for in-vivo determination of bio chemical concentrations comprising an optical illumination source and a detector, said detector characterized as including at least one pressure transducer having a geometric shape which results in a point within a test tissue being more strongly coupled than immediately surrounding points.

2) Apparatus of claim 1, said detector is further characterized as a directional microphone whereby a predetermined space is favored over other spatial regions with respect to transducer response and performance.

3) Apparatus of claim 2, said detector being arranged to address a portion of flesh contain interstitial fluids in human tissue.

4) Apparatus of claim 3, said bio chemical is a blood analyte.

5) Apparatus of claim 2, said detector is comprised of a plurality of transducers to form a multi-element detector array.

6) Apparatus of claim 5, said plurality of transducers are arranged in an axially symmetric pattern.

7) Apparatus of claim 6, said arrangement of transducers substantially forms an annulus.

8) Apparatus of claim 6, said arrangement of transducers a plurality of annuli at least one concentric with another.

9) Apparatus of claim 8, said detector includes electronic means of effecting a phase delay of signals generated at either of said transducers.

10) Apparatus of claim 8) said arrangement of transducers includes an optical port at the center.

11) Apparatus of claim 8, each ring is bifurcated into a plurality of wedge section sub-elements.

12) Apparatus of claim 11, detector includes electronic means of effecting a phase delay of signals generated at either of said sub-elements.

13) Methods of in-vivo determination of a bio chemical concentration by photoacoustic spectroscopy comprising the steps:
illuminating a tissue sample with middle infrared light;
receiving pressure waves emanating from illuminated tissue at a skin surface interface;
converting those pressure waves into electronic signals at a detector shaped such that energy emitted from a particular point is coupled to the transducer with a greater efficiency than other nearby points.

14) Methods of claim 13, said converting pressure waves into electronic signals step is done at a plurality of spatially removed locations.

15) Methods of claim 14, conversion of pressure waves is done simultaneously, thereafter a phase delay created electronically and introduced into at least one of the electronic signals.

16) Methods of 14, said converting pressure waves is done at a plurality of areas distributed about a symmetry axis.

17) Methods of claim 16, conversion of pressure waves is done simultaneously, thereafter a phase delay created electronically and introduced into at least one of the electronic signals.

18) Methods of claim 13, said steps include an intermediate step after receiving pressure waves at a skin surface comprising: reflecting said pressure waves from a shaped reflection surface whereby said pressure waves further propagate to a focus and become concentrated thereat.
